(19) United States
(12) Patent Application Publication
(10) Pub. No.: US 2015/0230769 A1
(43) Pub. Date: Aug. 20, 2015

(54) IMAGE DIAGNOSIS APPARATUS, CONTRAST AGENT INJECTOR, CONTRAST ENHANCED IMAGING AIDING APPARATUS, AND IMAGE DIAGNOSIS SYSTEM

(71) Applicants: Kabushiki Kaisha Toshiba, Minato-ku (JP); Toshiba Medical Systems Corporation, Otawara-shi (JP)

(72) Inventor: Masaharu TSUYUKI, Nasushiobara (JP)

(73) Assignees: Kabushiki Kaisha Toshiba, Minato-ku (JP); Toshiba Medical Systems Corporation, Otawara-shi (JP)

(21) Appl. No.: 14/620,654

(22) Filed: Feb. 12, 2015

(30) Foreign Application Priority Data
Feb. 12, 2014 (JP) 2014-024675

(51) Int. Cl. A61B 6/00; A61B 6/03; A61B 5/00; A61B 5/055

(52) U.S. Cl. CPC A61B 6/481; A61B 5/055; A61B 6/032; A61B 6/037; A61B 5/0037; A61B 6/488

(57) ABSTRACT

An image diagnosis apparatus according to an embodiment includes a judging unit and an output controlling unit. The judging unit judges whether an image taking protocol related to an image taking process performed on a subject and an injection protocol related to the administration of the contrast agent to the subject are consistent with each other, the injection protocol being set by a contrast agent injector configured to administer a contrast agent to the subject. When it has been determined that the injection protocol and the image taking protocol are not consistent with each other, the output controlling unit causes a predetermined output unit to output information indicating that the injection protocol and the image taking protocol are not consistent with each other.

### CROSS-REFERENCE TO RELATED APPLICATIONS

This application is based upon and claims the benefit of priority from Japanese Patent Application No. 2014-024675, filed on Feb. 12, 2014, the entire contents of all of which are incorporated herein by reference.

### FIELD

Embodiments described herein relate generally to an image diagnosis apparatus, a contrast agent injector, a contrast enhanced imaging aiding apparatus, and an image diagnosis system.

### BACKGROUND

Conventionally, with image diagnosis apparatuses such as ultrasound diagnosis apparatuses, X-ray Computed Tomography (CT) apparatuses, X-ray diagnosis apparatuses, Magnetic Resonance Imaging (MRI) apparatuses, and nuclear medical diagnosis apparatuses, a medical examination method is known by which, for the purpose of achieving an excellent contrast to render an organ or the like in an image taken of an examined subject, a contrast agent is administered (injected) to the subject by using a contrast agent injector.

For the medical examination method by which a contrast agent is administered, examples of methods for determining the timing with which images are taken (hereinafter, “image taking timing”) includes a bolus tracking method and a test bolus method. The bolus tracking method requires performing the image taking process only once. In contrast, according to the test bolus method, after a test administration of a contrast agent and an image taking process (a test scan) are performed, a main administration of the contrast agent and a main image taking process are performed. Thus, the image taking process needs to be performed twice.

However, because both an image diagnosis apparatus and a contrast agent injector are each able to have a setting with a protocol including a test bolus or with a protocol including no test bolus, it is inconvenient that consistency needs to be checked between an image taking protocol on the image diagnosis apparatus side and an injection protocol on the contrast agent injector side. For example, let us assume that a test administration of a contrast agent and an image taking process have been performed while a protocol including a test bolus process is selected for both the image diagnosis apparatus and the contrast agent injector. When it becomes necessary, after that, to change the image taking protocol, and when an image taking protocol including a test bolus process is selected, a conflict will occur between the injection protocol indicating that the main administration of the contrast agent is performed and the image taking protocol indicating that an image taking process (a test scan) related to a test administration of the contrast agent and the main image taking process are performed. As explained herein, it is not easy to set the image taking protocol and the injection protocol while maintaining the consistency therebetween.

### BRIEF DESCRIPTION OF THE DRAWINGS

FIG. 1 is a block diagram of an overall configuration of an X-ray CT system according to a first embodiment;

FIG. 2 is a block diagram of an example of a detailed configuration of a contrast agent injector according to the first embodiment;

FIG. 3 is a flowchart of an example of an operation performed by an X-ray CT apparatus;

FIG. 4 is a drawing for explaining inputting information to the X-ray CT apparatus and to the contrast agent injector, as well as communication between the X-ray CT apparatus and the contrast agent injector;

FIG. 5 is a drawing for explaining examples of an injection protocol and an image taking protocol;

FIG. 6 is a drawing for explaining another example of an image taking protocol;

FIG. 7 is a drawing for explaining an example of a display screen;

FIG. 8 is a drawing for explaining an example of a correction made on the image taking protocol;

FIG. 9 is a drawing for explaining another example of an injection protocol;

FIG. 10 is a drawing for explaining another example of the display screen;

FIG. 11 is a drawing for explaining an example of a correction made on the injection protocol;

FIG. 12 is a drawing for explaining an example in which an injection protocol and an image taking protocol are not consistent with each other;

FIG. 13 is a drawing for explaining yet another example of the display screen;

FIG. 14 is a drawing for explaining an example of a correction made on an injection protocol;

FIG. 15 is a block diagram of an exemplary configuration of a system controlling unit according to the first embodiment;

FIG. 16A is a flowchart of Steps S100-S127 of the operation in a scan process performed by the X-ray CT apparatus;

FIG. 16B is a flowchart of Steps S128-S138 of the operation in the scan process performed by the X-ray CT apparatus;

FIG. 17 is a block diagram of an example of a detailed configuration of a contrast agent injector according to a second embodiment; and

FIG. 18 is a block diagram of an overall configuration of an X-ray CT system according to a third embodiment.

### DETAILED DESCRIPTION

Exemplary embodiments of an image diagnosis apparatus, a contrast agent injector, a contrast enhanced imaging aiding apparatus, and an image diagnosis system will be explained in detail below, with reference to the accompanying drawings. In the following explanations, some of the constituent elements that are the same as one another will be referred to by using the same reference characters, and duplicated explanations will be omitted.

The image diagnosis apparatus may be, for example, an ultrasound diagnosis apparatus, an X-ray CT apparatus, an X-ray diagnosis apparatus, a magnetic resonance imaging apparatus, a nuclear medical diagnosis apparatus, or the like. The exemplary embodiments below will be explained by using an X-ray CT apparatus as an example.

An image diagnosis apparatus according to an embodiment includes a judging unit and an output controlling unit. The judging unit judges whether an image taking protocol related to an image taking process performed on a subject and an injection protocol related to the administration of the contrast agent to the subject are consistent with each other, the injection protocol being set by a contrast agent injector configured to administer a contrast agent to the subject. When it has been determined that the injection protocol and the image taking protocol are not consistent with each other, the output controlling unit causes a predetermined output unit to output information indicating that the injection protocol and the image taking protocol are not consistent with each other.

### First Embodiment

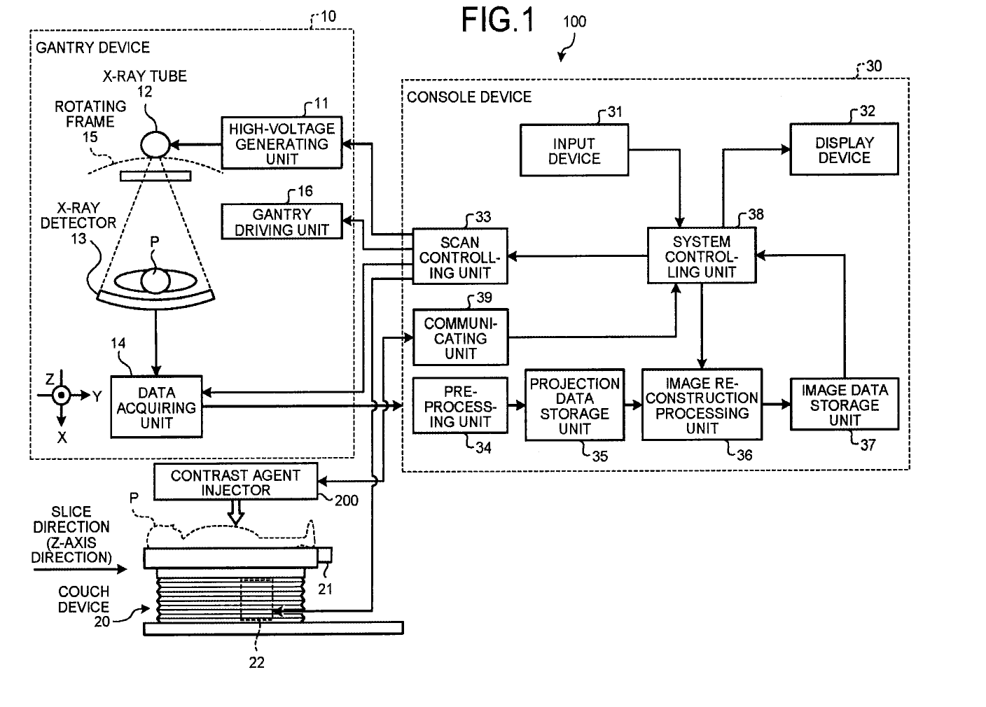

FIG. 1 is a block diagram of an overall configuration of an X-ray CT system according to a first embodiment. As illustrated in FIG. 1, the X-ray CT system includes an X-ray CT apparatus 100 and a contrast agent injector 200. The X-ray CT apparatus 100 includes a gantry device 10, a couch device 20, and a console device 30. The X-ray CT apparatus 100 and the contrast agent injector 200 are connected to each other so as to be able to communicate with each other via a communicating unit 39 included in the console device 30.

The X-ray CT apparatus 100 is configured to perform a tomography image taking process on an examined subject P, by radiating X-rays onto the subject P placed inside the gantry device 10 and performing a helical scan, a step-and-shoot scan, or the like. The contrast agent injector 200 is configured to administer a contrast agent to the subject P, in synchronization with the scan performed by the X-ray CT apparatus 100, while the X-ray CT apparatus 100 is performing the tomography image taking process. More specifically, into the X-ray CT apparatus 100, an image taking protocol (an image taking condition) is set, the image taking protocol being used for performing the scan (e.g., the helical scan, the step-and-shoot scan) by administering the contrast agent from the contrast agent injector 200. Further, into the contrast agent injector 200, an injection protocol (an injection plan) is set, the injection protocol being used for administering the contrast agent in synchronization with the image taking protocol performed by the X-ray CT apparatus 100.

In this situation, the image taking protocol describes settings for each scan such as a dosage, a scan speed, and a slice thickness, as well as a phase in which only a helical/step-and-shoot scan is performed, or phases indicating an image taking order in which a test scan and a helical/step-and-shoot scan are performed. Further, the injection protocol describes phases indicating an administering order in which a contrast agent and a physiological saline solution (PS) are administered, in synchronization with the image taking protocol.

As the image taking protocol and the injection protocol are set in this manner, the X-ray CT apparatus 100 is able to obtain CT images having an excellent contrast, by using the contrast agent administered by the contrast agent injector 200, while operating in synchronization with the administration of the contrast agent by the contrast agent injector 200.

The gantry device 10 is a device configured to radiate the X-rays onto the subject P and to acquire projection data. The gantry device 10 includes a high-voltage generating unit 11, an X-ray tube 12, an X-ray detector 13, a data acquiring unit 14, a rotating frame 15, and a gantry driving unit 16.

The high-voltage generating unit 11 is a device configured to supply a high voltage to the X-ray tube 12. The X-ray tube 12 is a vacuum tube configured to generate the X-rays by using the high voltage supplied by the high-voltage generating unit 11.

The X-ray detector 13 is a detector configured to detect X-rays that have passed through the subject P. More specifically, the X-ray detector 13 is a multi-row area detector structured by arranging, in a slice direction (the Z-axis direction illustrated in FIG. 1), a plurality of (e.g., 320) detecting element rows each made up of X-ray detecting elements corresponding to a plurality of channels. The X-ray detector 13 is capable of simultaneously detecting the X-rays that have passed through the subject P in a large area such as an area including an image taking target site (e.g., the heart, the brain, etc.).

The data acquiring unit 14 is a device configured to generate the projection data by using the X-rays detected by the X-ray detector 13.

The rotating frame 15 is an annular frame configured to rotate continuously at a high speed. The rotating frame 15 supports the X-ray tube 12 and the X-ray detector 13 so as to oppose each other while the subject P is interposed therebetween. The gantry driving unit 16 is a driving device configured to cause the X-ray tube 12 and the X-ray detector 13 to rotate on a circular trajectory centered on the subject P, by driving the rotating frame 15 to rotate.

The couch device 20 is a device on which the subject P is placed and includes a couchtop 21 and a couch driving device 22. The couchtop 21 is a plate on which the subject P is placed during an image taking process. The couch driving device 22 is configured to move the couchtop 21 in the slice direction.

The console device 30 is a device configured to receive various types of instructions related to operations performed on the X-ray CT apparatus 100 from a user such as a medical doctor or a medical technician and to reconstruct images from the projection data acquired by the gantry device 10. The console device 30 includes an input device 31, a display device 32, a scan controlling unit 33, a pre-processing unit 34, a projection data storage unit 35, an image reconstruction processing unit 36, an image data storage unit 37, a system controlling unit 38, and the communicating unit 39.

The input device 31 is a device that includes a mouse, a keyboard, and the like and is configured to receive instructions for the X-ray CT apparatus 100 from the user. For example, prior to an image taking process, the input device 31 receives subject information and a setting of an image taking protocol and also receives a setting of a Region Of Interest (ROI) to be set in a taken image. Further, during an image taking process, the input device 31 may receive an instruction to cancel a currently-used image taking protocol and to set and start another image taking protocol.

In this situation, examples of image taking protocols that can be set by the user include: a scan mode indicating a helical scan, a step-and-shoot scan, or the like; the presence/absence of an administration of a contrast agent performed by the contrast agent injector 200; and a method for determining the image taking timing when a contrast agent is to be administered.

Examples of methods for determining the image taking timing when a contrast agent is to be administered include a bolus tracking method and a test bolus method. When the bolus tracking method is selected, an image taking protocol is set so as to perform an image taking process once per administration of a contrast agent. When the test bolus method is selected, an image taking protocol is set so as to perform an image taking process (a test scan) corresponding to a test administration of a contrast agent and to perform a main image taking process corresponding to a main administration of the contrast agent after calculating the image taking timing from the test scan.

The display device 32 is a device that includes a display using a Liquid Crystal Display (LCD) or the like and is configured to display various types of information. For example, the display device 32 displays image data stored in the image data storage unit 37 (explained later) and a Graphical User Interface (GUI) used for receiving various types of instructions from the user.

The scan controlling unit 33 is a processing unit configured, under the control of the system controlling unit 38 (explained later), to radiate the X-rays onto the subject P and to acquire the projection data, by driving the high-voltage generating unit 11, the data acquiring unit 14, the gantry driving unit 16, and the couch driving device 22, on the basis of the image taking protocol instructed by the system controlling unit 38.

The pre-processing unit 34 is a processing unit configured to perform a pre-processing process such as a sensitivity correction on the projection data generated by the data acquiring unit 14. The projection data storage unit 35 is a storage unit configured to store therein the projection data pre-processed by the pre-processing unit 34. The image reconstruction processing unit 36 is a processing unit configured, under the control of the system controlling unit 38 (explained later), to reconstruct image data from the projection data stored in the projection data storage unit 35. The image data storage unit 37 is a storage unit configured to store therein the image data reconstructed by the image reconstruction processing unit 36.

The system controlling unit 38 is a processing unit configured to exercise overall control of the X-ray CT apparatus 100, by controlling operations of the gantry device 10, the couch device 20, and the console device 30. More specifically, the system controlling unit 38 includes a Central Processing Unit (CPU), a Read-Only Memory (ROM), and a Random Access Memory (RAM) and is configured to realize the control over the X-ray CT apparatus 100, as a result of the CPU performing software processing by loading a computer program stored in the ROM into the RAM and sequentially executing the computer program. By controlling the various functional units, the system controlling unit 38 is configured to radiate the X-rays onto the subject P either continuously or intermittently on the basis of the image taking protocol and to reconstruct the images by detecting the X-rays that have passed through the subject P.

In that situation, the RAM included in the system controlling unit 38 functions as a storage unit that stores therein one or more items in the image taking process that have already been performed according to the image taking protocol. For example, when a test scan corresponding to a test administration of a contrast agent has been finished, the RAM included in the system controlling unit 38 stores therein information indicating that a test scan has already been performed. More specifically, the RAM included in the system controlling unit 38 stores therein information indicating that a test scan has already been performed, for each of different types of medical examinations with respect to test administrations (for each type of contrast agent and for each site of the subject P to which a contrast agent is administered).

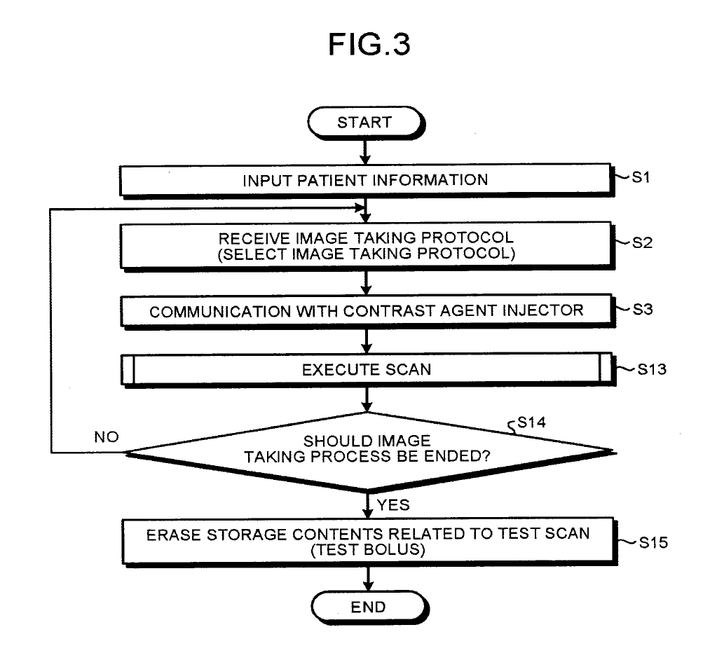

Further, as illustrated in FIG. 15, the system controlling unit 38 according to the first embodiment includes a setting unit 38a, a judging unit 38b, an output controlling unit 38c, and a protocol correcting unit 38d. Details of the functional units included in the system controlling unit 38 will be explained later, with reference to FIG. 3. FIG. 15 is a block diagram of an exemplary configuration of the system controlling unit 38 according to the first embodiment.

Under the control of the system controlling unit 38, the communicating unit 39 is configured to communicate with the contrast agent injector 200 connected thereto via a communication cable or the like through a Controller Area Network (CAN) or the like. More specifically, the communicating unit 39 communicates with the contrast agent injector 200 according to the specifications of CiA 425 of the CAN. With this arrangement, the X-ray CT apparatus 100 and the contrast agent injector 200 transmit and receive a synchronization signal to and from each other at the start of an image taking process and an administration of a contrast agent. Further, the X-ray CT apparatus 100 is also able to read the injection protocol of the contrast agent injector 200 and to set an injection protocol therein.

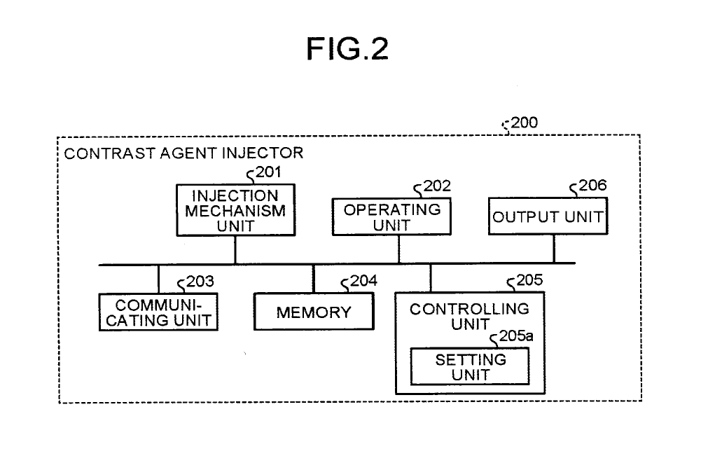

FIG. 2 is a block diagram of an example of a detailed configuration of the contrast agent injector 200. As illustrated in FIG. 2, the contrast agent injector 200 includes an injection mechanism unit 201, an operating unit 202, a communicating unit 203, a memory 204, a controlling unit 205, and an output unit 206.

The injection mechanism unit 201 includes a mechanism for administering the contrast agent to the subject P. The mechanism includes a power source that is able to change various conditions such as an injection amount, an injection speed, an injection pressure, and the like. A commonly-used power source is a motor. The power source may be a pump or the like that has a variable pneumatic or hydraulic pressure level.

The operating unit 202 is configured to receive an input of subject information designated by an operation by the user and input information related to the administration of the contrast agent such as the injection protocol. In the contrast agent injector 200, the input information that is related to the administration of the contrast agent such as the injection protocol and that is received by the operating unit 202 as an input is stored and set into the memory 204, under the control of the controlling unit 205.

In this situation, the injection protocol includes information indicating whether the bolus tracking method (a contrast agent is administered once) or the test bolus method (a contrast agent is administered twice: the test administration and the main administration) is to be used, as well as a type of the medical examination, an injection amount of the contrast agent for each injection, and an injection speed. Examples of types of medical examinations include types of sites of the subject P and types of contrast agents. Examples of the types of sites include a blood vessel, the urinary tract, the head, the abdomen, and a joint. Further, examples of types of contrast agents that can be used in X-ray image taking processes include positive contrast agents (e.g., barium sulfate, an iodine preparation) that have a larger X-ray attenuation coefficient than the tissues of the subject P in the surroundings thereof and negative contrast agents (e.g., gas such as air, oxygen, carbon dioxide, or nitrogen) that have a smaller X-ray attenuation coefficient than the tissues of the subject P in the surroundings thereof.

The output unit 206 includes a display device using an LCD or the like and is configured to display various types of information. For example, the output unit 206 displays a GUI used for receiving various types of instructions from the user including the input information related to the administration of the contrast agent such as the image taking protocol and the injection protocol received from the user.

The communicating unit 203 is configured to communicate with the X-ray CT apparatus 100 in synchronization therewith. The memory 204 is configured to store therein the input information received by the operating unit 202 as an input and information received by the communicating unit 203.

The controlling unit 205 is configured to control operations of the contrast agent injector 200. More specifically, the controlling unit 205 includes a CPU, a ROM, and a RAM and is configured to realize the control over the contrast agent injector 200 as a result of the CPU performing software processing by loading a computer program stored in the ROM into the RAM and sequentially executing the computer program. Even more specifically, the controlling unit 205 controls the synchronization with the X-ray CT apparatus 100 and the injection of the contrast agent to the subject P by the injection mechanism unit 201, so that the administration of the contrast agent is performed according to the injection protocol stored in the memory 204. Further, the controlling unit 205 includes a setting unit 205a, as illustrated in FIG. 2. The setting unit 205a is configured to set the input information into the memory 204, the input information being related to the administration of the contrast agent such as the injection protocol received by the operating unit 202 as an input.

FIG. 3 is a flowchart of an example of an operation performed by the X-ray CT apparatus 100. As illustrated in FIG. 3, in the X-ray CT apparatus 100, when a process is started, an input of subject information is received via the input device 31 (step S1).

Subsequently, in the X-ray CT apparatus 100, an image taking protocol is received via the input device 31, so that the received image taking protocol is set therein (step S2). In this situation, the setting unit 38a included in the system controlling unit 38 sets the image taking protocol that is received via the input device 31 and is related to the image taking process performed on the subject P. For example, the setting unit 38a included in the system controlling unit 38 stores the image taking protocol into the RAM included in the system controlling unit 38. To set the image taking protocol in this situation, one of a plurality of image taking protocols that are set in the ROM or the like in advance may be selected. In that situation, the setting unit 38a stores the selected image taking protocol into the RAM included in the system controlling unit 38.

At that time, when an image taking process that uses a contrast agent is to be performed, an image taking protocol indicating either the bolus tracking method or the test bolus method is set as a method for determining image taking timing. Further, the setting unit 205a included in the contrast agent injector 200 receives an injection protocol via the operating unit 202 and sets the received injection protocol. In other words, the setting unit 205a included in the contrast agent injector 200 sets the injection protocol related to the administration of the contrast agent to the subject P. To set the injection protocol, one of a plurality of injection protocols that are set in the ROM or the like in advance may be selected.

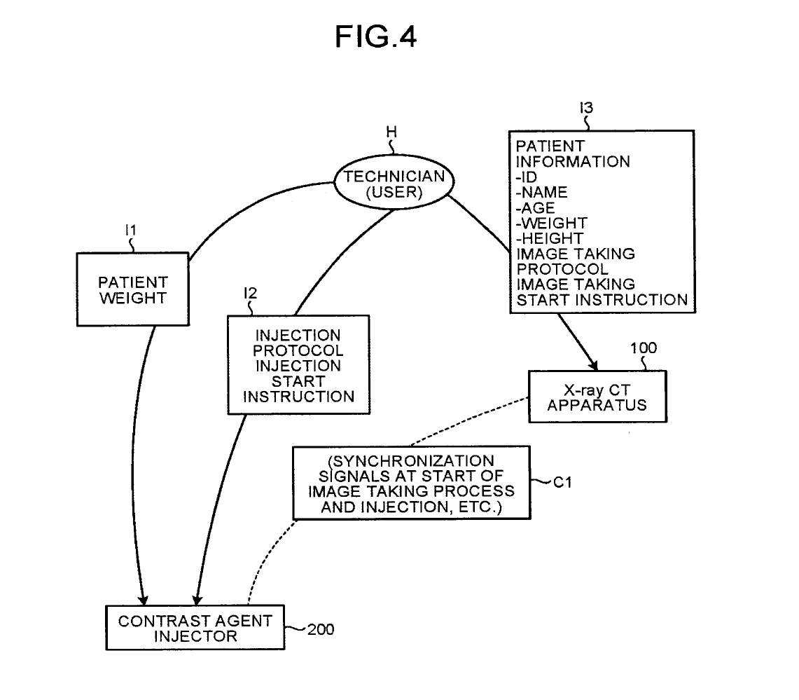

FIG. 4 is a drawing for explaining inputting information to the X-ray CT apparatus 100 and to the contrast agent injector 200, as well as communication between the X-ray CT apparatus 100 and the contrast agent injector 200. As illustrated in FIG. 4, when an image taking process that uses a contrast agent is to be performed, a medical technician H (a user) inputs input information I1 including the weight of the subject to be referred to for calculating an injection amount and input information I2 including an injection protocol and an injection start instruction, to the contrast agent injector 200. Further, the medical technician H inputs input information I3 including subject information such as an ID, the name, the age, the weight, and the height, an image taking protocol, and an image taking start instruction, to the X-ray CT apparatus 100. By receiving the inputs of the injection start instruction and the image taking start instruction, the X-ray CT apparatus 100 and the contrast agent injector 200 start a tomography image taking process in synchronization with the administration of the contrast agent, by performing a communication C1 to provide each other with synchronization signals related to the start of the image taking process and the injection.

Subsequently, the X-ray CT apparatus 100 performs a communication with the contrast agent injector 200, via the communicating unit 39 (step S3). As a result of the communication, the X-ray CT apparatus 100 reads and obtains the injection protocol that is set in the contrast agent injector 200.

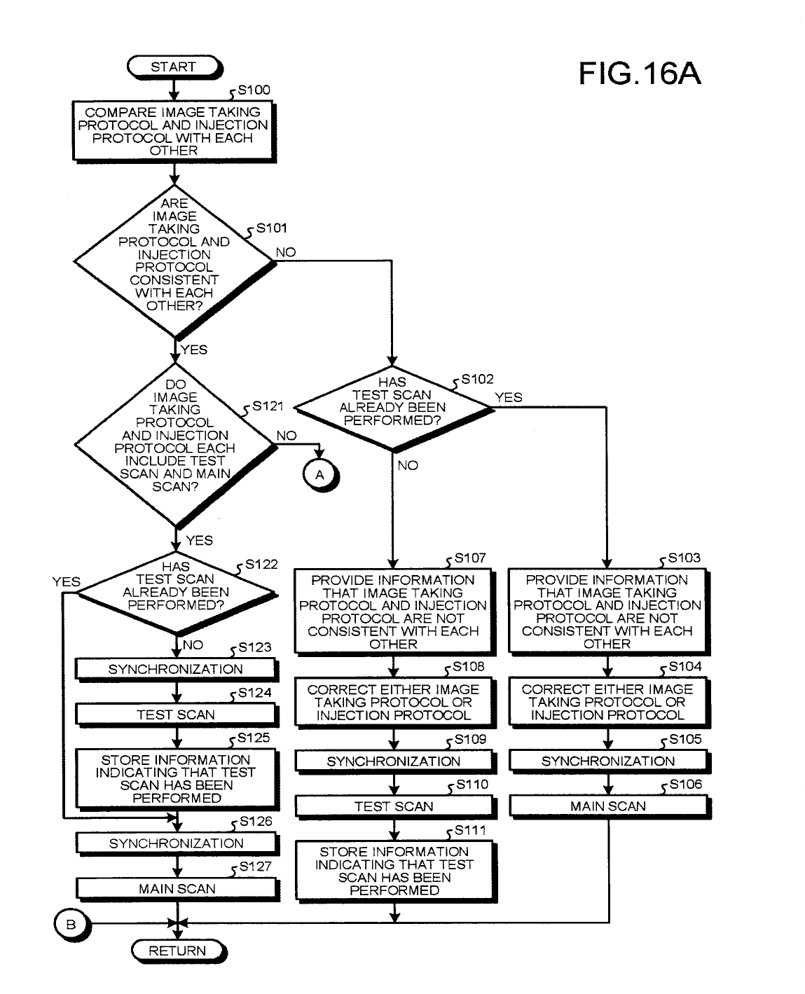

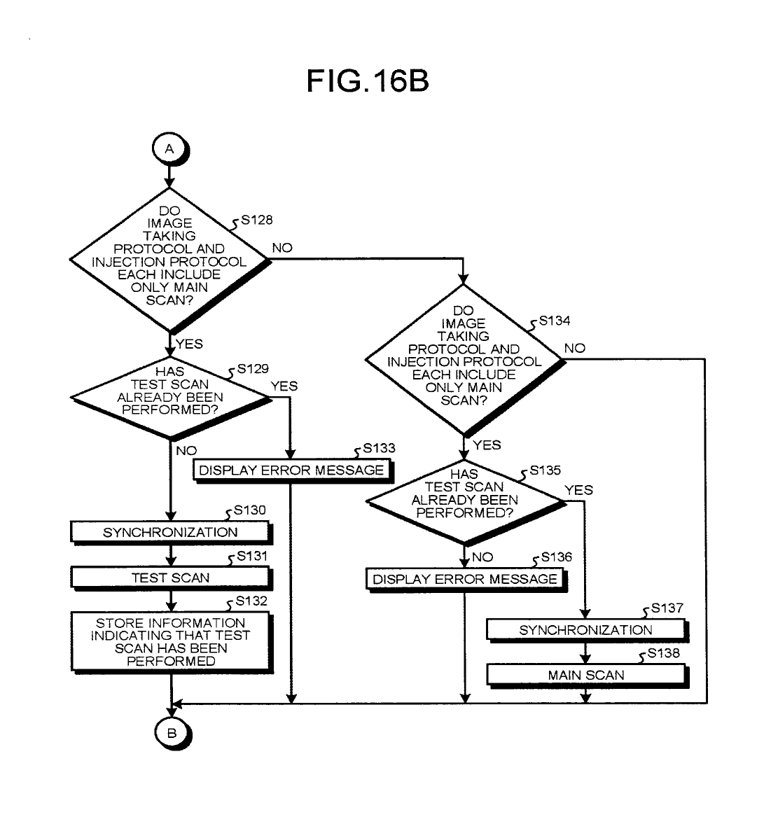

After that, the judging unit 38b included in the system controlling unit 38 performs a scan process (step S13). An operation in the scan process performed by the X-ray CT apparatus 100 will be explained, with reference to FIGS. 16A and 16B. FIG. 16A is a flowchart of Steps S100-S127 of the operation in the scan process performed by the X-ray CT apparatus 100. FIG. 16B is a flowchart of Steps S128-S138 of the operation in the scan process performed by the X-ray CT apparatus. The processing procedure illustrated in FIGS. 16A and 16B corresponds to the scan process at step S13 in FIG. 3.

As illustrated in FIGS. 16A and 16B, the judging unit 38b included in the system controlling unit 38 compares the image taking protocol and the injection protocol with each other (step S100). At step S101, the judging unit 38b included in the system controlling unit 38 judges whether the image taking protocol and the injection protocol that are set are consistent with each other, on the basis of the presence/absence of a test administration in the injection protocol that is set and the presence/absence of a test scan corresponding to the test administration in the image taking protocol that is set. In other words, the judging unit 38b included in the system controlling unit 38 judges whether the injection protocol of the contrast agent injector obtained via the communicating unit 39 and the image taking protocol that is set are consistent with each other.

More specifically, the judging unit 38b included in the system controlling unit 38 determines that the injection protocol and the image taking protocol are consistent with each other (step S101: Yes), when the presence/absence of a test administration in the injection protocol set in the contrast agent injector 200 is consistent with the presence/absence of a test scan in the image taking protocol set in the X-ray CT apparatus 100, including the type of the medical examination (the type of the contrast agent and the site of the subject P to which the contrast agent is administered). On the contrary, the judging unit 38b included in the system controlling unit 38 determines that the image taking protocol and the injection protocol that are set are not consistent with each other (step S9: No), when an injection protocol including a test administration is set in the contrast agent injector 200, while an image taking protocol that includes no test scan corresponding to the test administration (no test scan having the same type of contrast agent and the same site of the subject P to which the contrast agent is administered) is set in the X-ray CT apparatus 100. Also, the judging unit 38b included in the system controlling unit 38 determines that the image taking protocol and the injection protocol that are set are not consistent with each other (step S9: No), when an image taking protocol including a test scan is set in the X-ray CT apparatus 100, while an injection protocol that includes no test administration corresponding to the test scan is set in the contrast agent injector 200.

When the image taking protocol and the injection protocol that are set are consistent with each other (step S9: Yes), the system controlling unit 38 proceeds to the process at step S121. On the contrary, when the image taking protocol and the injection protocol that are set are not consistent with each other (step S101: No), the judging unit 38b included in the system controlling unit 38 judges whether a test scan has been performed, by referring to the storage contents of the RAM, which is configured to store therein one or more items in the image taking process that have already been performed according to the image taking protocol (step S102).

More specifically, because the RAM included in the system controlling unit 38 stores therein information indicating that a test scan has already been performed, for each of different types of medical examinations with respect to test administrations (for each type of contrast agent and for each site of the subject P to which a contrast agent is administered), it is judged at step S102 whether or not such a test scan has already been performed that has the same conditions regarding the type of the contrast agent and the site to which the contrast agent is administered, with respect to either the test administration included in the injection protocol that is set or the test scan included in the image taking protocol that is set.

When the test scan has already been performed (step S102: Yes), the output controlling unit 38c included in the system controlling unit 38 causes the display device 32 to display information indicating that the image taking protocol and the injection protocol are not consistent with each other, so as to notify the user (step S103). In other words, when it has been determined that the image taking protocol and the injection protocol are not consistent with each other, the output controlling unit 38c causes the display device 32 to output the information indicating that the image taking protocol and the injection protocol are not consistent with each other.

After the process at step S103, to arrange the image taking protocol and the injection protocol to be consistent with each other, the protocol correcting unit 38d included in the system controlling unit 38 corrects one of the image taking protocol and the injection protocol so as to match the other (step S104). In other words, when it has been determined that the injection protocol and the image taking protocol are not consistent with each other, the protocol correcting unit 38d corrects one of the injection protocol and the image taking protocol so as to match the other. More specifically, when an instruction to correct either the image taking protocol or the injection protocol is received via the input device 31 after the user is notified that the image taking protocol and the injection protocol are not consistent with each other, the protocol correcting unit 38d included in the system controlling unit 38 makes the correction so as to arrange one of the protocols to match the other, in such a manner that the presence/absence of a test scan in the image taking protocol is consistent with the presence/absence of a test administration in the injection protocol, including the type of the medical examination (the type of the contrast agent and the site of the subject P to which the contrast agent is administered). In one example, when the injection protocol and the image taking protocol are not consistent with each other, and also, a test scan has already been performed, the protocol correcting unit 38d deletes the test scan from one of the protocols. In this situation, when the protocol correcting unit 38d corrects the injection protocol set in the contrast agent injector 200, the setting unit 205a included in the contrast agent injector 200 sets the corrected injection protocol into the memory 204. In contrast, when the protocol correcting unit 38d corrects the image taking protocol set in the X-ray CT apparatus 100, the setting unit 38a included in the X-ray CT apparatus 100 sets the corrected image taking protocol into the RAM included in the system controlling unit 38.

After that, the system controlling unit 38 synchronizes with the contrast agent injector 200 (step S105) and instructs the scan controlling unit 33 to perform the main scan (step S106). For example, the system controlling unit 38 instructs the scan controlling unit 33 to use the image taking protocol stored in the RAM included in the system controlling unit 38. Accordingly, the scan controlling unit 33 acquires image taking data on the basis of the image taking protocol that is set. In this situation, when either the image taking protocol or the injection protocol was corrected at step S104, the scan controlling unit 33 acquires the image taking data on the basis of the corrected image taking protocol. For example, when the image taking protocol was corrected at step S104, the scan controlling unit 33 acquires the image taking data on the basis of the corrected image taking protocol that was obtained by correcting the image taking protocol so as to match the injection protocol. After the process at step S106 is finished, the system controlling unit 38 ends the scan process and proceeds to step S14 in FIG. 3.

When the test scan has not yet been performed (step S102: No), the output controlling unit 38c included in the system controlling unit 38 causes the display device 32 to display information indicating that the image taking protocol and the injection protocol are not consistent with each other, so as to notify the user (step S107). In other words, when it has been determined that the image taking protocol and the injection protocol are not consistent with each other, the output controlling unit 38c causes the display device 32 to output the information indicating that the image taking protocol and the injection protocol are not consistent with each other.

After the process at step S107, to arrange the image taking protocol and the injection protocol to be consistent with each other, the protocol correcting unit 38d included in the system controlling unit 38 corrects one of the image taking protocol and the injection protocol so as to match the other (step S108). In other words, when it has been determined that the injection protocol and the image taking protocol are not consistent with each other, the protocol correcting unit 38d corrects one of the injection protocol and the image taking protocol so as to match the other. More specifically, when an instruction to correct either the image taking protocol or the injection protocol is received via the input device 31 after the user is notified that the image taking protocol and the injection protocol are not consistent with each other, the protocol correcting unit 38d included in the system controlling unit 38 makes the correction so as to arrange one of the protocols to match the other, in such a manner that the presence/absence of a test scan in the image taking protocol is consistent with the presence/absence of a test administration in the injection protocol, including the type of the medical examination (the type of the contrast agent and the site of the subject P to which the contrast agent is administered). In one example, when the injection protocol and the image taking protocol are not consistent with each other, and also, a test scan has not yet been performed, the protocol correcting unit 38d deletes the main scan from one of the protocols. In this situation, when the protocol correcting unit 38d corrects the injection protocol set in the contrast agent injector 200, the setting unit 205a included in the contrast agent injector 200 sets the corrected injection protocol into the memory 204. In contrast, when the protocol correcting unit 38d corrects the image taking protocol set in the X-ray CT apparatus 100, the setting unit 38a included in the X-ray CT apparatus 100 sets the corrected image taking protocol into the RAM included in the system controlling unit 38.

The system controlling unit 38 synchronizes with the contrast agent injector 200 (step S109) and instructs the scan controlling unit 33 to perform the test scan (step S110). Accordingly, the scan controlling unit 33 performs the test scan on the basis of the image taking protocol. Subsequently, the system controlling unit 38 stores information indicating that the test scan has been performed (the test scan is finished) into the RAM included in the system controlling unit 38 (step S111). After the process at step S111 is finished, the system controlling unit 38 ends the scan process and proceeds to step S14 in FIG. 3.

When the judging unit 38b included in the system controlling unit 38 determines that the injection protocol and the image taking protocol are consistent with each other (step S101: Yes), the judging unit 38b judges whether the image taking protocol and the injection protocol each include a test scan and a main scan (step S121). In this situation, when the judging unit 38b included in the system controlling unit 38 has determined that the image taking protocol and the injection protocol each include a test scan and a main scan (step S121: Yes), the judging unit 38b further judges whether the test scan has already been performed (step S122). When the judging unit 38b included in the system controlling unit 38 determines that the test scan has already been performed (step S122: Yes), the system controlling unit 38 proceeds to step S126.

On the contrary, when the judging unit 38b included in the system controlling unit 38 does not determine that the test scan has already been performed (step S122: No), the system controlling unit 38 synchronizes with the contrast agent injector 200 (step S123) and instructs the scan controlling unit 33 to perform the test scan (step S124). Accordingly, the scan controlling unit 33 performs the test scan on the basis of the image taking protocol. Subsequently, the system controlling unit 38 stores information indicating that the test scan has been performed (the test scan is finished) into the RAM included in the system controlling unit 38 (step S125).

After that, the system controlling unit 38 synchronizes with the contrast agent injector 200 (step S126) and instructs the scan controlling unit 33 to perform the main scan (step S127). For example, the system controlling unit 38 instructs the scan controlling unit 33 to use the image taking protocol stored in the RAM included in the system controlling unit 38. Accordingly, the scan controlling unit 33 acquires image taking data on the basis of the image taking protocol that is set. After the process at step S127 is finished, the system controlling unit 38 ends the scan process and proceeds to step S14 in FIG. 3.

In contrast, when the judging unit 38b included in the system controlling unit 38 does not determine at step S121 that the image taking protocol and the injection protocol each include a test scan and a main scan (step S121: No), the judging unit 38b further judges whether the image taking protocol and the injection protocol each include only a test scan (step S128). When the judging unit 38b included in the system controlling unit 38 has determined that the image taking protocol and the injection protocol each include only a test scan (step S128: Yes), the judging unit 38b further judges whether the test scan has already been performed (step S129).

When the judging unit 38b included in the system controlling unit 38 does not determine that the test scan has already been performed (step S129: No), the system controlling unit 38 synchronizes with the contrast agent injector 200 (step S130) and instructs the scan controlling unit 33 to perform the test scan (step S131). Accordingly, the scan controlling unit 33 performs the test scan on the basis of the image taking protocol. Subsequently, the system controlling unit 38 stores information indicating that the test scan has been performed (the test scan is finished) into the RAM included in the system controlling unit 38 (step S132). After the process at step S132 is finished, the system controlling unit 38 ends the scan process and proceeds to step S14 in FIG. 3.

On the contrary, when the judging unit 38b included in the system controlling unit 38 determines that the test scan has already been performed (step S129: Yes), the output controlling unit 38c included in the system controlling unit 38 causes the display device 32 to display an error message indicating that the test scan is selected despite that the test scan has already been performed (step S133). After the process at step S133 is finished, the system controlling unit 38 ends the scan process and proceeds to step S14 in FIG. 3.

In contrast, when the judging unit 38b included in the system controlling unit 38 does not determine at step S128 that the image taking protocol and the injection protocol each include only a test scan (step S128: No), the judging unit 38b further judges whether the image taking protocol and the injection protocol each include only a main scan (step S134). When the judging unit 38b included in the system controlling unit 38 has determined that the image taking protocol and the injection protocol each include only a main scan (step S134: Yes), the judging unit 38b further judges whether the test scan has already been performed (step S135).

When the judging unit 38b included in the system controlling unit 38 does not determine that the test scan has already been performed (step S135: No), the output controlling unit 38c included in the system controlling unit 38 causes the display device 32 to display an error message indicating that the test scan for performing the main scan has not yet been performed (step S136). After the process at step S136 is finished, the system controlling unit 38 ends the scan process and proceeds to step S14 in FIG. 3.

On the contrary, when the judging unit 38b included in the system controlling unit 38 determines that the test scan has already been performed (step S135: Yes), the system controlling unit 38 synchronizes with the contrast agent injector 200 (step S137) and instructs the scan controlling unit 33 to perform the main scan (step S138). Accordingly, similarly to the process at step S127, the scan controlling unit 33 acquires image taking data on the basis of the image taking protocol that is set. After the process at step S138 is finished, the system controlling unit 38 ends the scan process and proceeds to step S14 in FIG. 3.

In contrast, when the judging unit 38b included in the system controlling unit 38 does not determine at step S134 that the image taking protocol and the injection protocol each include only a main scan (step S134: No), the scan process is ended so that the process proceeds to step S14 in FIG. 3.

Returning to the description of FIG. 3, subsequent to the process at step S13, the system controlling unit 38 judges whether the image taking process performed on the subject P should be ended or not, by judging whether an ending instruction or the like is input via the input device 31 (step S14). When the image taking process should not be ended (step S14: No), because the image taking process performed on the subject P is continued, the system controlling unit 38 returns to the process at step S2. On the contrary, when the image taking process should be ended (step S14: Yes), because the image taking process performed on the subject P is finished, the storage contents of the RAM related to the test scan (the test bolus process) are erased (step S15), and the process is thus ended. As a result, the storage contents indicating that the test scan has already been performed are held only while the image taking process is continued for the single subject P.

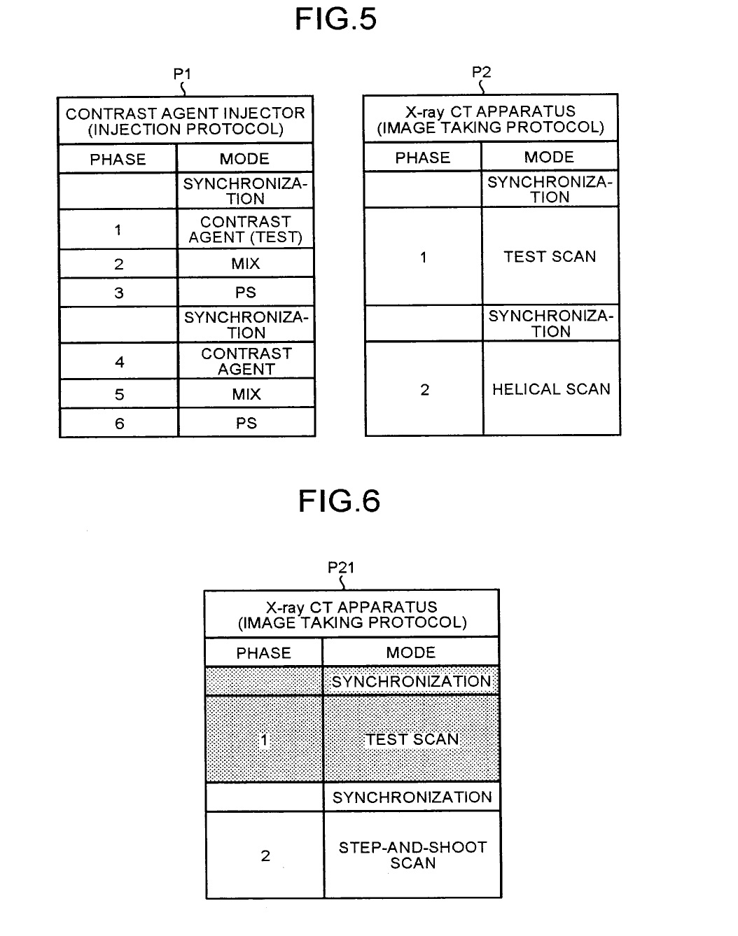

Next, the operation of the X-ray CT apparatus 100 described in the flowchart above will be further explained, with a number of specific examples of injection protocols and image taking protocols. FIG. 5 is a drawing for explaining examples of an injection protocol P1 and an image taking protocol P2. As illustrated in FIG. 5, the injection protocol P1 is an example of a protocol in which a test bolus process including a test administration of a contrast agent is performed. The image taking protocol P2 is an example of a protocol in which a helical scan is performed by administering a contrast agent, and also, as a method for determining the image taking timing, a test bolus process is performed (with a test scan). In this situation, let us assume that the injection protocol P1 is set in the contrast agent injector 200, whereas the image taking protocol P2 is set in the X-ray CT apparatus 100. In that situation, the judging unit 38b determines at step S101 that the injection protocol P1 and the image taking protocol P2 are consistent with each other.

FIG. 6 is a drawing for explaining an example of an image taking protocol P21. The image taking protocol P21 is an example of a protocol in which a step-and-shoot scan is performed by administering a contrast agent, and also, as a method for determining the image taking timing, a test bolus process is performed (with a test scan).

In this situation, let us assume that, after the test scan is performed with the settings of the injection protocol P1 and the image taking protocol P2 illustrated in FIG. 5, the X-ray CT apparatus 100 has received an instruction to cancel the currently-used image taking protocol P2 and to set and start the image taking protocol P21. In other words, let us assume that the user has made a change from a helical scan to a step-and-shoot scan after the test scan. In that situation, because the image taking protocol P21 that includes the test scan (in the phase indicated with the hatching) is set despite that the test scan has already been performed, the judgment result at step S102 is in the positive (step S102: Yes), so that a warning is displayed (step S103).

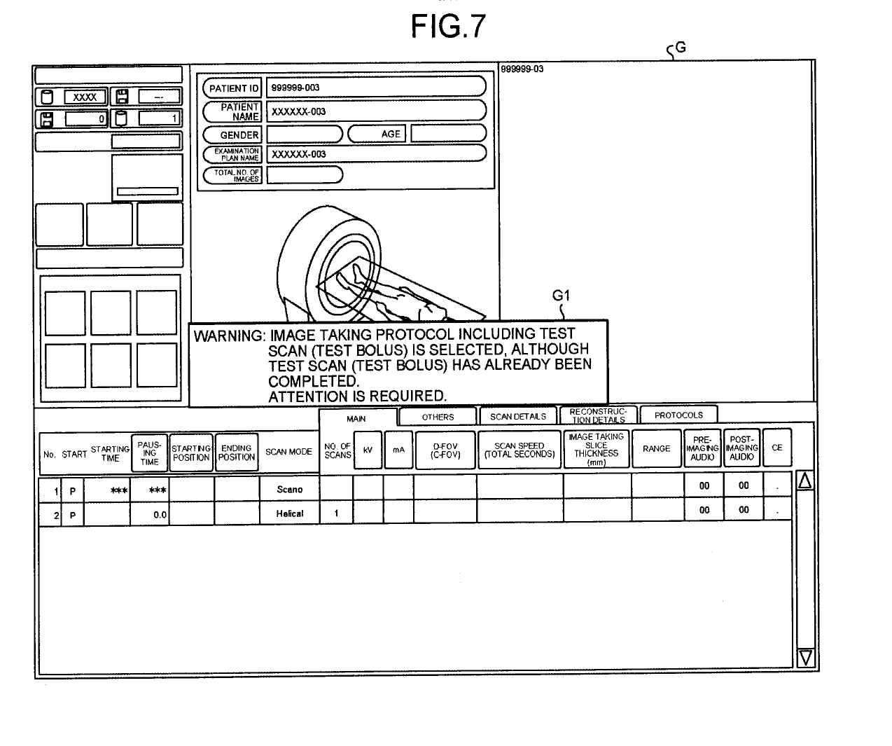

FIG. 7 is a drawing for explaining an example of a display screen G. When the image taking protocol P2 has been changed to the image taking protocol P21, the display screen G of the display device 32 displays, as illustrated in FIG. 7, a warning display G1 so as to indicate that the image taking protocol P21 that includes the test scan is selected despite that the test scan has already been performed (step S103). Accordingly, the user is able to easily recognize that the image taking protocol P21 that includes the test scan is selected despite that the test scan is finished and that a conflict (an inconsistency) has occurred with the injection protocol P1 indicating the main administration of the contrast agent. Consequently, even when the image taking protocol and the injection protocol are inconsistent with each other, the user is able to easily redo the settings so as to maintain consistency.

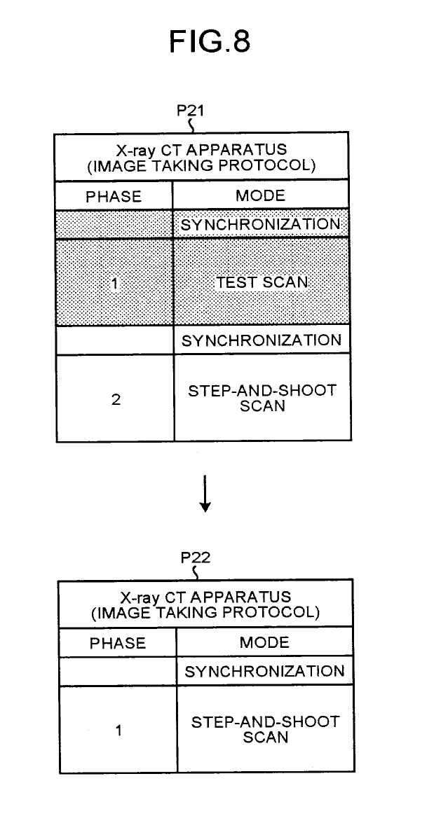

FIG. 8 is a drawing for explaining an example of a correction made on the image taking protocol P21. When a correction instruction is received via the input device 31 after the warning display G1 is displayed, the image taking protocol P21 that includes the test scan is corrected at step S104, to be an image taking protocol P22 that includes no test scan, as illustrated in FIG. 8. To make this correction, another arrangement is acceptable in which, while the image taking protocol P21 that includes the test scan remains unchanged, the starting position is corrected to be placed after the test scan (the starting position is corrected so as to be in the next phase following the phase indicated with the hatching in FIG. 8). Accordingly, even when the currently-used image taking protocol P2 is cancelled so as to set another image taking protocol, the user is able to easily set the image taking protocol that is consistent with the injection protocol P1.

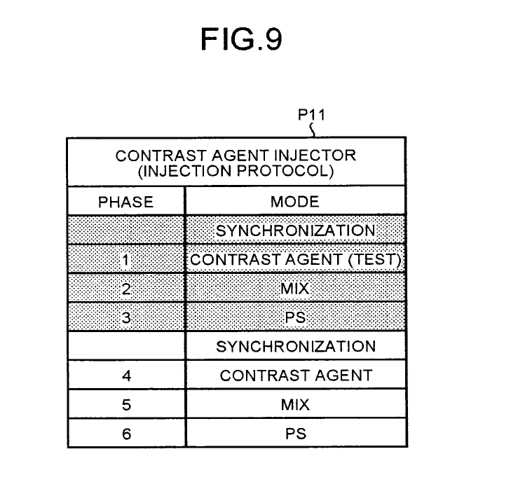

FIG. 9 is a drawing for explaining an example of an injection protocol P11. The injection protocol P11 is an example of a protocol in which a test bolus process including a test administration of a contrast agent is performed. In this situation, let us assume that, after the test scan is performed with the settings of the injection protocol P1 and the image taking protocol P2 illustrated in FIG. 5, the contrast agent injector 200 has received an instruction to cancel the currently-used image taking protocol P2 and to set and start the injection protocol P11.

In that situation, after the test scan, because the user sets the injection protocol P11 that includes the test administration (in the phase indicated with the hatching) despite that the test scan has already been performed (the test administration is finished), the judgment result at step S102 is in the positive (step S102: Yes), so that a warning is displayed (step S103).

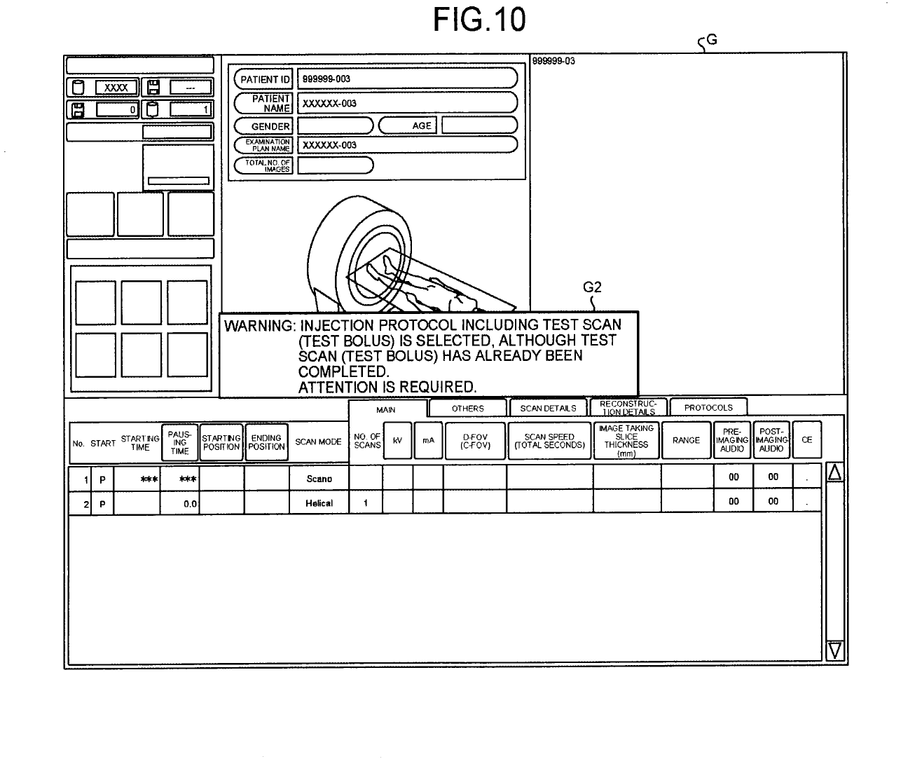

FIG. 10 is a drawing for explaining another example of the display screen G. When the injection protocol P1 has been changed to the injection protocol P11, the display screen G of the display device 32 displays, as illustrated in FIG. 10, a warning display G2 so as to indicate that the image taking protocol P11 corresponding to the test bolus process is selected despite that the test scan has already been performed (step S103). Accordingly, the user is able to easily recognize that the injection protocol P11 corresponding to the test bolus process is selected despite that the test scan is finished and that a conflict (an inconsistency) has occurred with the image taking protocol P2 indicating that a main image taking process is performed in correspondence with a main administration of the contrast agent. Consequently, even when the image taking protocol and the injection protocol are inconsistent with each other, the user is able to easily redo the settings so as to maintain consistency.

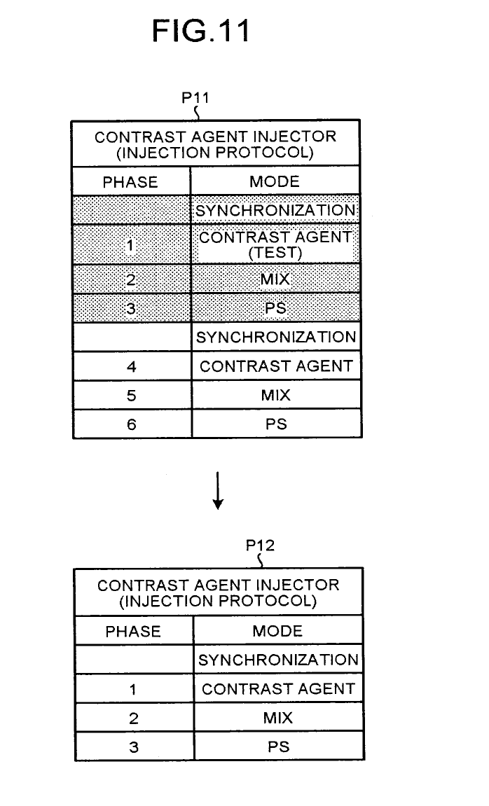

FIG. 11 is a drawing for explaining an example of a correction made on the injection protocol P11. When a correction instruction is received via the input device 31 after the warning display G2 is displayed, the protocol correcting unit 38d corrects, at step S104, the injection protocol P11 that includes the test administration, to be an injection protocol P12 that includes no test administration, as illustrated in FIG. 11. To make this correction, another arrangement is acceptable in which, while the injection protocol P11 that includes the test administration remains unchanged, the starting position is corrected to be placed after the test administration (the starting position is corrected so as to be in the next phase following the phase indicated with the hatching in FIG. 11). Accordingly, even when the currently-used image taking protocol P2 is cancelled so as to set another injection protocol, the user is able to easily set the injection protocol that is consistent with the image taking protocol P2.

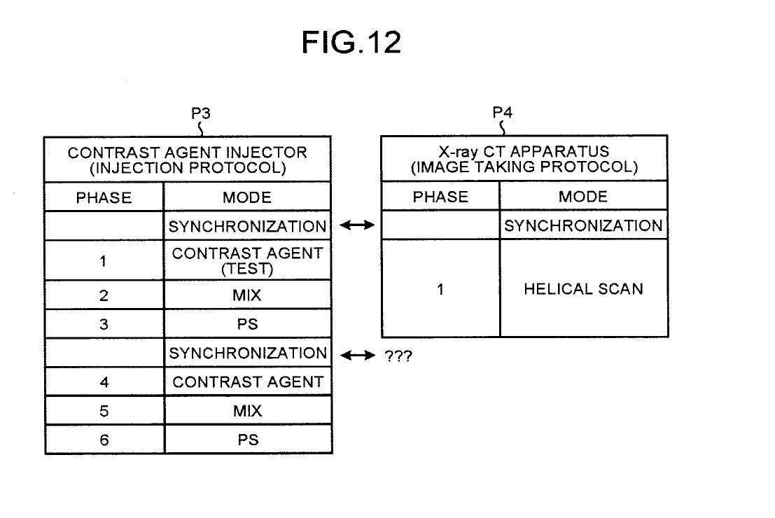

FIG. 12 is a drawing for explaining an example in which an injection protocol P3 and an image taking protocol P4 are not consistent with each other. As illustrated in FIG. 12, the injection protocol P3 is an example of a protocol in which a test bolus process including a test administration of a contrast agent is performed. The image taking protocol P4 is an example of a protocol in which a helical scan is performed without administering any contrast agent. The injection protocol P3 and the image taking protocol P4 are not consistent with each other, as apparent when being compared side by side, because the presence/absence of the test administration and the presence/absence of the test scan do not match. In this situation, let us assume that the injection protocol P3 is set into the contrast agent injector 200, whereas the image taking protocol P4 is set into the X-ray CT apparatus 100. In that situation, the judging unit 38b determines at step S101 that the injection protocol P3 and the image taking protocol P4 are not consistent with each other. Further, because the test scan for performing the main scan has not yet been performed, the judgment result at step S102 is in the negative (step S102: No), so that the processes at steps S107 is performed.

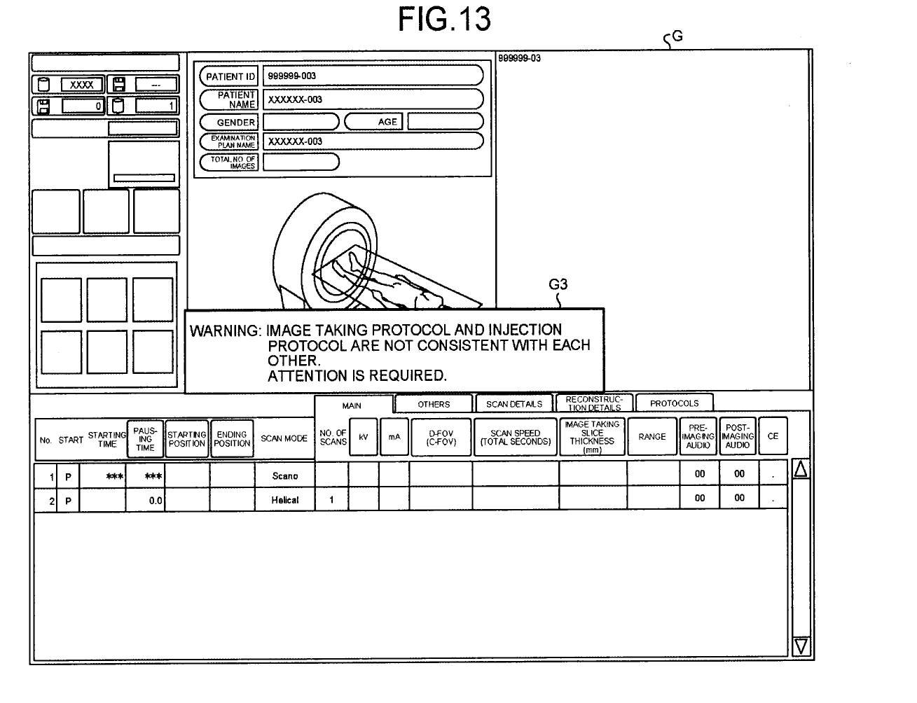

FIG. 13 is a drawing for explaining yet another example of the display screen G. When protocols are not consistent with each other like the injection protocol P3 and the image taking protocol P4, the display screen G of the display device 32 displays, as illustrated in FIG. 13, a warning display G3 at step S107 so as to indicate that the protocols are not consistent with each other. Accordingly, the user is able to easily recognize that a conflict (an inconsistency) has occurred between the injection protocol P3 and the image taking protocol P4. Consequently, even when the injection protocol P3 and the image taking protocol P4 are inconsistent with each other, the user is able to easily redo the settings so as to maintain consistency.

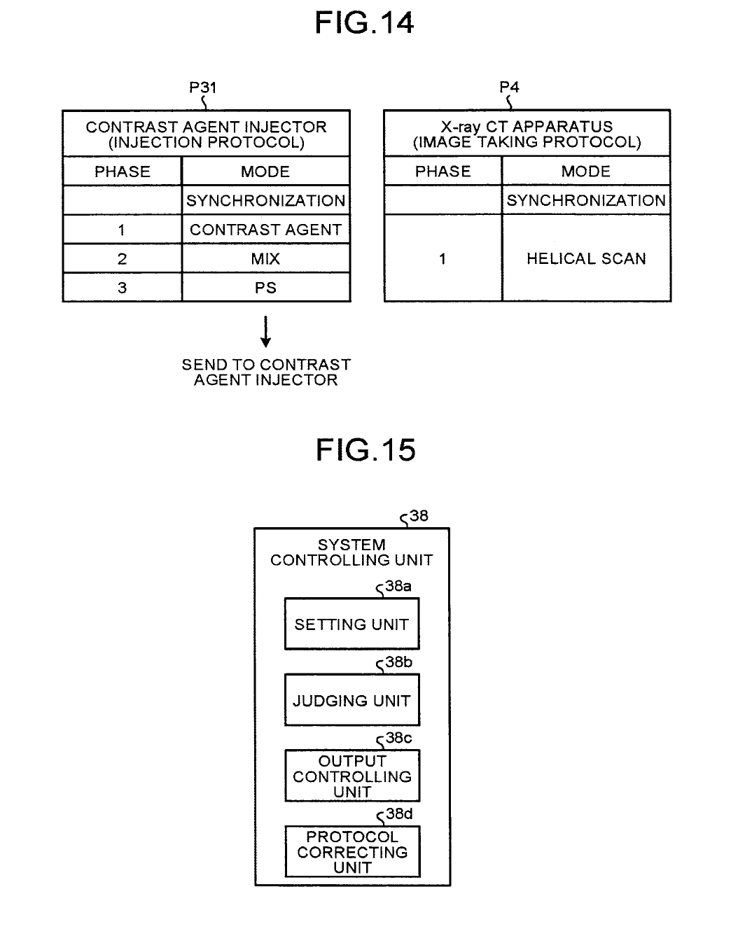

FIG. 14 is a drawing for explaining an example of a correction made on an injection protocol. When a correction instruction is received via the input device 31 after the warning display G3 is displayed, the X-ray CT apparatus 100 sends an injection protocol P31 that includes no test administration to the contrast agent injector 200, as illustrated in FIG. 14, so as to correct the setting in the contrast agent injector 200. In the example illustrated in FIG. 14, the correction is made so as to use the injection protocol P31 that includes no test administration; however, needless to say, it is also acceptable to make a correction so as to use an image taking protocol that includes a test scan. Accordingly, even when the injection protocol P3 and the image taking protocol P4 are inconsistent with each other, the user is able to easily make a correction so as to obtain the settings that are consistent with each other.

As explained above, in the X-ray CT system according to the first embodiment, even when both the X-ray CT apparatus 100 and the contrast agent injector 200 are each able to have the setting with a protocol including a test bolus process or with a protocol including no test bolus process, the user is able to easily configure the settings while maintaining consistency, without worrying about the consistency regarding the presence/absence of the test bolus processes. For example, even when only an image taking protocol indicating that a test bolus process is performed is set on the X-ray CT apparatus 100 side, when an injection protocol that includes no test bolus is set on the contrast agent injector 200 side, the image taking protocol is corrected to be an image taking protocol including no test scan, so as to maintain consistency. Accordingly, on the X-ray CT apparatus 100 side, there is no need to selectively address the situations with the presence/absence of the test bolus process. (There is no need to prepare image taking protocols and injection protocols in two types, i.e., one that includes a test bolus process and the other that includes no test bolus process.) It is therefore possible to improve operability.

Further, in the first embodiment described above, the example is explained in which, when the image taking protocol and the injection protocol that are set are not consistent with each other, the system controlling unit 38 causes the display device 32 to display the information indicating that the injection protocol and the image taking protocol are not consistent with each other; however, possible embodiments are not limited to this example. For instance, the system controlling unit 38 may cause the output unit 206 included in the contrast agent injector 200 to output information indicating that the injection protocol and the image taking protocol are not consistent with each other. In that situation, the system controlling unit 38 generates the information indicating that the injection protocol and the image taking protocol are not consistent with each other and transmits the generated information to the contrast agent injector 200 via the communicating unit 39. After that, the controlling unit 205 included in the contrast agent injector 200 causes the output unit 206 to display the information received from the system controlling unit 38 via the communicating unit 203.

Further, in the embodiments described above, the example is explained in which, after the warning is displayed (step S103) or the information indicating that the protocols are not consistent with each other is displayed (step S107), when a correction instruction is received via the input device 31, either the image taking protocol or the injection protocol is corrected (step S104, S108). However, another arrangement is acceptable in which either the image taking protocol or the injection protocol is immediately corrected (step S104, S108), without involving the process of displaying the warning (step S103) or displaying the information indicating that the protocols are not consistent with each other (step S107). More specifically, the system controlling unit 38 may automatically correct either the image taking protocol or the injection protocol without particularly displaying the warning (step S103) or displaying the information indicating that the protocols are not consistent with each other (step S107) and without receiving any correction instruction from the console device 30. In that situation, it is possible to correct either the image taking protocol or the injection protocol more easily, without involving the confirmation from the user or the operation of the correction instruction.

### Second Embodiment

In the first embodiment described above, the example is explained in which, in the X-ray CT system, the X-ray CT apparatus 100 judges whether the injection protocol and the image taking protocol are consistent with each other. In this regard, the process of judging whether the injection protocol and the image taking protocol are consistent with each other may be performed by a constituent element other than the X-ray CT apparatus 100. Thus, in a second embodiment, an example will be explained in which, in the X-ray CT system, a contrast agent injector judges whether an injection protocol and an image taking protocol are consistent with each other.

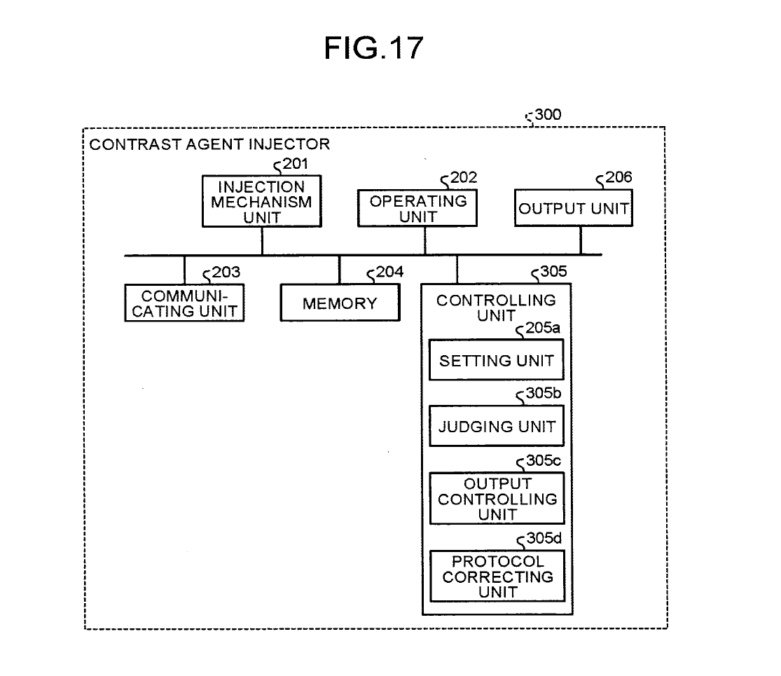

The configuration of the X-ray CT system according to the second embodiment is the same as the configuration of the X-ray CT system according to the first embodiment illustrated in FIG. 1, except that the configuration of a controlling unit included in the contrast agent injector is different. Thus, in the second embodiment, only the configuration of the contrast agent injector will be explained. In the second embodiment, the system controlling unit 38 included in the X-ray CT apparatus 100 either does not use the functions of the judging unit 38b, the output controlling unit 38c, and the protocol correcting unit 38d or is configured without including the judging unit 38b, the output controlling unit 38c, and the protocol correcting unit 38d. FIG. 17 is a block diagram of an example of a detailed configuration of a contrast agent injector 300 according to the second embodiment.

As illustrated in FIG. 17, the contrast agent injector 300 according to the second embodiment includes the injection mechanism unit 201, the operating unit 202, the communicating unit 203, the memory 204, the output unit 206, and a controlling unit 305. Some of the functional units illustrated in FIG. 17 that have the same functions as those illustrated in FIG. 2 will be referred to by using the same reference characters, and detailed explanation thereof will be omitted. The communicating unit 203 is configured, under the control of the controlling unit 305, to communicate with the X-ray CT apparatus 100 connected thereto via a communication cable or the like through a CAN or the like. More specifically, the communicating unit 203 communicates with the X-ray CT apparatus 100 according to the specifications of CiA 425 of the CAN. With this arrangement, the X-ray CT apparatus 100 and the contrast agent injector 300 transmit and receive a synchronization signal to and from each other at the start of an image taking process and an administration of a contrast agent. Further, the contrast agent injector 300 is also able to read the image taking protocol of the X-ray CT apparatus 100 and to set an image taking protocol therein.

As illustrated in FIG. 17, the controlling unit 305 includes the setting unit 205a, a judging unit 305b, an output controlling unit 305c, and a protocol correcting unit 305d. Like the setting unit 205a included in the contrast agent injector 200 according to the first embodiment, the setting unit 205a is configured to set an injection protocol related to administering a contrast agent to the subject P.

The judging unit 305b has the same functions as those of the judging unit 38b. In other words, similarly to the processes at steps S102 illustrated in FIGS. 16A and 16B, the judging unit 305b is configured to judge whether an injection protocol indicating that a test administration is performed or an image taking protocol indicating that a test scan is performed is set or not, despite that the test scan has already been performed. Further, similarly to the process performed by the judging unit 38b at step S101 illustrated in FIGS. 16A and 16B, the judging unit 305b is configured to judge whether the image taking protocol set in the X-ray CT apparatus obtained via the communicating unit 203 and the injection protocol that is set are consistent with each other.

The output controlling unit 305c has the same functions as those of the output controlling unit 38c. In other words, similarly to the process at step S103 illustrated in FIGS. 16A and 16B, the output controlling unit 305c is configured to cause the output unit 206 to output a warning display, when either an injection protocol indicating that a test administration is performed or an image taking protocol indicating that a test scan is performed is set despite that the test scan has already been performed. Further, when it has been determined that the injection protocol and the image taking protocol are not consistent with each other, the output controlling unit 305c is configured to cause the output unit 206 to display information indicating that the injection protocol and the image taking protocol are not consistent with each other, similarly to the process performed by the output controlling unit 38c at step S107 illustrated in FIGS. 16A and 16B.

The protocol correcting unit 305d has the same functions as those of the protocol correcting unit 38d. In other words, similarly to the process at step S104 illustrated in FIGS. 16A and 16B, the protocol correcting unit 305d is configured to correct either an image taking protocol or an injection protocol that includes a test scan, to be an image taking protocol or an injection protocol that includes no test scan. Further, when it has been determined that the injection protocol and the image taking protocol are not consistent with each other, the protocol correcting unit 305d is configured to correct one of the injection protocol and the image taking protocol so as to match the other, similarly to the process performed by the protocol correcting unit 38d at step S108 illustrated in FIGS. 16A and 16B. In this situation, when the protocol correcting unit 305d corrects the image taking protocol set in the X-ray CT apparatus 100, the setting unit 38a included in the X-ray CT apparatus 100 sets the corrected image taking protocol into the RAM included in the system controlling unit 38. In contrast, when the protocol correcting unit 305d corrects the injection protocol set in the contrast agent injector 300, the setting unit 205a included in the contrast agent injector 300 sets the corrected injection protocol into the memory 204.

As explained above, in the X-ray CT system according to the second embodiment, the contrast agent injector 300 judges whether the injection protocol and the image taking protocol are consistent with each other. Consequently, in the X-ray CT system according to the second embodiment, even when both the X-ray CT apparatus 100 and the contrast agent injector 300 are each able to have the setting with a protocol including a test bolus process or with a protocol including no test bolus process, the user is able to easily configure the settings while maintaining consistency, without worrying about the consistency regarding the presence/absence of the test bolus processes.

Further, in the second embodiment, the example is explained in which the output controlling unit 305c causes the output unit 206 to display the information indicating that the injection protocol and the image taking protocol are not consistent with each other and the warning indicating that a test scan has already been performed; however, possible embodiments are not limited to this example. For instance, the output controlling unit 305c may cause the display device 32 to display the information indicating that the injection protocol and the image taking protocol are not consistent with each other and the warning indicating that a test scan has already been performed. In that situation, the output controlling unit 305c generates the information indicating that the injection protocol and the image taking protocol are not consistent with each other and the warning indicating that a test scan has already been performed and transmits the generated information and warning to the X-ray CT apparatus 100 via the communicating unit 203. After that, the system controlling unit 38 included in the X-ray CT apparatus 100 causes the display device 32 to display the information and the warning received from the output controlling unit 305c via the communicating unit 39.

### Third Embodiment

In the first and the second embodiments described above, the examples are explained in which either the X-ray CT apparatus 100 or the contrast agent injector 300 included in the X-ray CT system judges whether the injection protocol and the image taking protocol are consistent with each other. In this regard, the process of judging whether the injection protocol and the image taking protocol are consistent with each other may be performed by a constituent element other than the X-ray CT apparatus 100 and the contrast agent injector 300 included in the X-ray CT system. Thus, in a third embodiment, an example will be explained in which a contrast enhanced imaging aiding apparatus, which is an apparatus other than the X-ray CT apparatus 100 and the contrast agent injector 300, judges whether an injection protocol and an image taking protocol are consistent with each other.

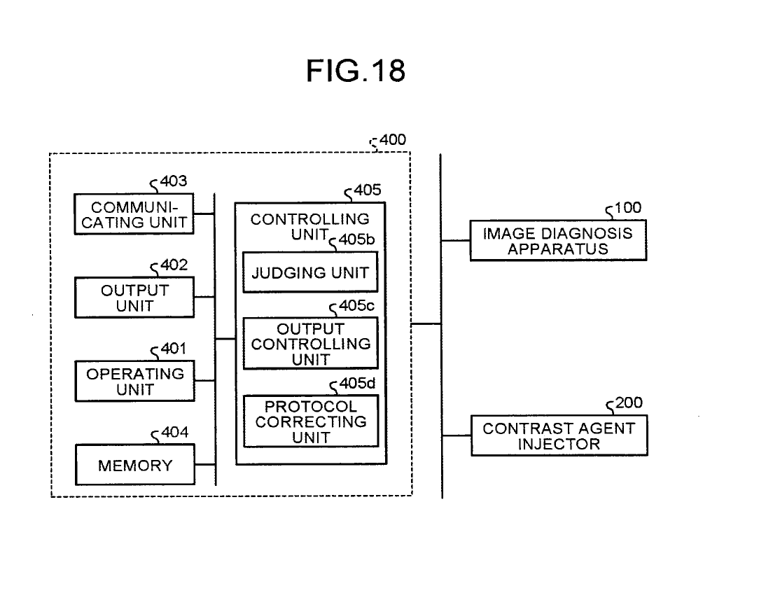

FIG. 18 is a block diagram of an overall configuration of an X-ray CT system according to the third embodiment. As illustrated in FIG. 18, an X-ray CT system according to the third embodiment includes the X-ray CT apparatus 100, the contrast agent injector 200, and a contrast enhanced imaging aiding apparatus 400. In the X-ray CT system according to the third embodiment, the contrast enhanced imaging aiding apparatus 400 is connected to the X-ray CT apparatus 100 and to the contrast agent injector 200 via a network. The X-ray CT apparatus 100 and the contrast agent injector 200 are the same as the X-ray CT apparatus 100 and the contrast agent injector 200 described in the first embodiment. Thus, detailed explanation thereof will be omitted. In the third embodiment, the system controlling unit 38 included in the image diagnosis apparatus 100 either does not use the functions of the judging unit 38b, the output controlling unit 38c, and the protocol correcting unit 38d or is configured without including the judging unit 38b, the output controlling unit 38c, and the protocol correcting unit 38d.

As illustrated in FIG. 18, the contrast enhanced imaging aiding apparatus 400 according to the third embodiment includes an operating unit 401, an output unit 402, a communicating unit 403, a memory 404, and a controlling unit 405. The operating unit 401 includes a mouse, a keyboard, and the like and is configured to receive instructions for the contrast enhanced imaging aiding apparatus 400 from the user. The output unit 402 includes a display device using an LCD or the like and is configured to display various types of information. For example, the output unit 402 displays a GUI used for receiving various types of instructions from the user and the various types of instructions received from the user. The memory 404 is configured to store therein input information received by the operating unit 401 as an input and information received by the communicating unit 403.

The communicating unit 403 is configured, under the control of the controlling unit 405, to communicate with the X-ray CT apparatus 100 and the contrast agent injector 200 connected thereto via a communication cable or the like through a CAN or the like. With this arrangement, the contrast enhanced imaging aiding apparatus 400 transmits and receives a synchronization signal to and from the X-ray CT apparatus 100 and the contrast agent injector 200 at the start of an image taking process and an administration of a contrast agent. Further, the contrast enhanced imaging aiding apparatus 400 is also able to read the image taking protocol of the X-ray CT apparatus 100, to set an image taking protocol therein, to read the injection protocol of the contrast agent injector 200, and to set a injection protocol therein. In other words, the communicating unit 403 obtains the image taking protocol that is related to the image taking process performed on the subject P and is set in the X-ray CT apparatus 100 from the X-ray CT apparatus 100, and further obtains the injection protocol that is related to the administration of the contrast agent to the subject P and is set in the contrast agent injector 200 from the contrast agent injector 200. Further, the communicating unit 403 causes an image taking protocol corrected by a protocol correcting unit 405d to be set into the X-ray CT apparatus 100 and causes an injection protocol corrected by the protocol correcting unit 405d to be set into the contrast agent injector 200.

As illustrated in FIG. 18, the controlling unit 405 includes a judging unit 405b, an output controlling unit 405c, and a protocol correcting unit 405d. The judging unit 405b has the same functions as those of the judging unit 38b. In other words, similarly to the processes at steps S102 illustrated in FIGS. 16A and 16B, the judging unit 405b is configured to judge whether an injection protocol indicating that a test administration is performed or an image taking protocol indicating that a test scan is performed is set or not, despite that the test scan has already been performed. Further, similarly to the process performed by the judging unit 38b at step S101 illustrated in FIGS. 16A and 16B, the judging unit 405b is configured to judge whether the injection protocol and the image taking protocol are consistent with each other.

The output controlling unit 405c has the same functions as those of the output controlling unit 38c. In other words, similarly to the process at step S103 illustrated in FIGS. 16A and 16B, the output controlling unit 405c is configured to cause the output unit 206 to output a warning display, when either an injection protocol indicating that a test administration is performed or an image taking protocol indicating that a test scan is performed is set despite that the test scan has already been performed. Further, when it has been determined that the injection protocol and the image taking protocol are not consistent with each other, the output controlling unit 405c is configured to cause the output unit 402 to display information indicating that the injection protocol and the image taking protocol are not consistent with each other, similarly to the process performed by the output controlling unit 38c at step S108 illustrated in FIGS. 16A and 16B.

The protocol correcting unit 405d has the same functions as those of the protocol correcting unit 38d. In other words, similarly to the process at step S104 illustrated in FIGS. 16A and 16B, the protocol correcting unit 405d is configured to correct either an image taking protocol or an injection protocol that includes a test scan, to be an image taking protocol or an injection protocol that includes no test scan. Further, when it has been determined that the injection protocol and the image taking protocol are not consistent with each other, the protocol correcting unit 405d is configured to correct one of the injection protocol and the image taking protocol so as to match the other, similarly to the process performed by the protocol correcting unit 38d at step S109 illustrated in FIGS. 16A and 16B. In this situation, when the protocol correcting unit 405d corrects the image taking protocol set in the X-ray CT apparatus 100, the protocol correcting unit 405d causes the corrected image taking protocol to be set into the X-ray CT apparatus 100 via the communicating unit 403. Accordingly, the setting unit 38a included in the X-ray CT apparatus 100 sets the corrected image taking protocol into the RAM included in the system controlling unit 38. In contrast, when the protocol correcting unit 405d corrects the injection protocol set in the contrast agent injector 200, the protocol correcting unit 405d causes the corrected injection protocol to be set into the contrast agent injector 200 via the communicating unit 403. Accordingly, the setting unit 205a included in the contrast agent injector 200 sets the corrected injection protocol into the memory 204.

As explained above, in the X-ray CT system according to the third embodiment, the contrast enhanced imaging aiding apparatus 400 judges whether the injection protocol and the image taking protocol are consistent with each other. Consequently, in the X-ray CT system according to the third embodiment, even when both the X-ray CT apparatus 100 and the contrast agent injector 200 are each able to have the setting with a protocol including a test bolus process or with a protocol including no test bolus process, the user is able to easily configure the settings while maintaining consistency, without worrying about the consistency regarding the presence/absence of the test bolus processes.

Further, in the third embodiment, the example is explained in which the output controlling unit 405c causes the output unit 402 to display the information indicating that the injection protocol and the image taking protocol are not consistent with each other and the warning indicating that a test scan has already been performed; however, possible embodiments are not limited to this example. For instance, the output controlling unit 405c may cause one or both of the display device 32 and the output unit 206 included in the contrast agent injector 200 to display the information indicating that the injection protocol and the image taking protocol are not consistent with each other and the warning indicating that a test scan has already been performed. In that situation, the output controlling unit 405c may further cause the output unit 402 to display the information indicating that the injection protocol and the image taking protocol are not consistent with each other and/or the warning indicating that a test scan has already been performed.

Further, in the third embodiment, the example is explained in which the communicating unit 403 obtains the image taking protocol from the X-ray CT apparatus 100 and obtains the injection protocol from the contrast agent injector 200; however, possible embodiments are not limited to this example. For instance, the communicating unit 403 may obtain the image taking protocol and the injection protocol obtained by the X-ray CT apparatus 100, from the X-ray CT apparatus 100. Alternatively, the communicating unit 403 may obtain the injection protocol and the image taking protocol obtained by the contrast agent injector 200, from the contrast agent injector 200.

### Other Embodiments

In the first to the third embodiments, the examples are explained in which the information indicating that the image taking protocol and the injection protocol are not consistent with each other and the warning indicating that a test scan has already been performed are displayed by one of the predetermined output units; however, possible embodiments are not limited to these examples. For example, the output controlling unit 38c (305c, 405c) may output audio indicating that the image taking protocol and the injection protocol are not consistent with each other and/or the warning indicating that a test scan has already been performed, or alternatively, may issue a notification with a buzzer sound or vibration, or may output information by turning on a warning light. In other words, when it has been determined that the injection protocol and the image taking protocol are not consistent with each other, the output controlling unit 38c (305c, 405c) causes the predetermined output unit to output the information indicating that the injection protocol and the image taking protocol are not consistent with each other. Further, when an injection protocol or an image taking protocol indicating that a test administration is performed is set despite that a test scan has already been performed, the output controlling unit 38c (305c, 405c) causes the predetermined output unit to output a warning indicating that a test scan has already been performed.

Further, in the first to the third embodiments described above, the examples are explained in which, when the image taking protocol and the injection protocol that are set are not consistent with each other in the X-ray CT system, the information indicating that the protocols are not consistent with each other is displayed, and also, either the image taking protocol or the injection protocol is corrected; however, possible embodiments are not limited these examples. For instance, when the image taking protocol and the injection protocol that are set are not consistent with each other, the process of correcting either the image taking protocol or the injection protocol may be omitted. In that situation, the information indicating that the protocols are not consistent with each other is displayed in the X-ray CT system, so that a correction to be made on either the image taking protocol or the injection protocol is received from the user. Alternatively, when the image taking protocol and the injection protocol that are set are not consistent with each other, either the image taking protocol or the injection protocol may automatically be corrected in the X-ray CT system, while the process of displaying the information indicating that the protocols are not consistent with each other is omitted.

Similarly, in the X-ray CT system, when an injection protocol indicating that a test administration is performed or an image taking protocol indicating that a test scan is performed is set despite that the test scan has already been performed, the process of correcting either the image taking protocol or the injection protocol may be omitted. In that situation, in the X-ray CT system, a warning indicating that a test scan has already been performed is displayed, so as to receive a correction to be made on either the image taking protocol or the injection protocol from the user. Alternatively, in the X-ray CT system, when an injection protocol indicating that a test administration is performed or an image taking protocol indicating that a test scan is performed is set despite that the test scan has already been performed, either the image taking protocol or the injection protocol may automatically be corrected, without causing the predetermined output unit to output the warning indicating that a test scan has already been performed.

Further, in the exemplary embodiments above, the examples are explained in which either the image taking protocol or the injection protocol is obtained by performing a communication through the CAN or the like; however, possible embodiments are not limited to these examples. For instance, the X-ray CT apparatus 100 may store a plurality of injection protocols in addition to a plurality of image taking protocols in a ROM or the like in advance, so as to obtain an identifier indicating the injection protocol that is set from the contrast agent injector 200. Similarly, the contrast agent injector 300 may store a plurality of image taking protocols in addition to a plurality of injection protocols in a ROM or the like in advance, so as to obtain an identifier indicating the image taking protocol that is set from the X-ray CT apparatus 100. Further, the contrast enhanced imaging aiding apparatus 400 may store a plurality of image taking protocols and a plurality of injection protocols in a ROM or the like in advance, so as to obtain an identifier indicating the image taking protocol that is set and an identifier indicating the injection protocol that is set. Alternatively, in the X-ray CT system, instead of obtaining the identifier indicating the image taking protocol that is set or the identifier indicating the injection protocol that is set, the image taking protocol that is set and/or the injection protocol that is set may be received from the user.

Further, in the exemplary embodiments above, the example is explained in which, when the helical scan is performed by administering the contrast agent, the currently-used image taking protocol is cancelled and changed to the step-and-shoot scan; however, possible embodiments are not limited to this example. For instance, another situation is also applicable where, when a step-and-shoot scan is performed by administering a contrast agent, the currently-used image taking protocol is cancelled and changed to a helical scan.

According to at least one aspect of the embodiments described above, it is possible to easily set the protocols while maintaining the consistency between the image taking protocol and the injection protocol.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An image diagnosis apparatus comprising:
a judging unit configured to judge whether an image taking protocol related to an image taking process performed on a subject and an injection protocol related to the administration of the contrast agent to the subject are consistent with each other, the injection protocol being set by a contrast agent injector configured to administer a contrast agent to the subject; and
an output controlling unit configured, when it has been determined that the injection protocol and the image taking protocol are not consistent with each other, to cause a predetermined output unit to output information indicating that the injection protocol and the image taking protocol are not consistent with each other.

2. The image diagnosis apparatus according to claim 1, wherein
the injection protocol includes a presence/absence of a test administration,
the image taking protocol includes a presence/absence of a test scan corresponding to the test administration, and
the judging unit judges whether the injection protocol and the image taking protocol are consistent with each other, on a basis of the presence/absence of the test administration in the injection protocol and the presence/absence of the test scan corresponding to the test administration in the image taking protocol.

3. The image diagnosis apparatus according to claim 2, further comprising: a storage unit configured, when the test scan corresponding to the test administration has been performed on the subject, to store therein information indicating that the test scan has already been performed, wherein
when the injection protocol includes the test administration or when the image taking protocol includes the test scan, the judging unit judges whether the test scan has already been performed or not, and
when it has been determined that the test scan has already been performed, the output controlling unit causes the predetermined output unit to output information indicating either that the injection protocol including the test administration is set despite that the test administration has already been performed or that the image taking protocol including the test scan is set despite that the test scan has already been performed.

4. The image diagnosis apparatus according to claim 3, wherein
the storage unit stores therein the information indicating that the test scan has already been performed, for each of conditions regarding a type of the contrast agent and a site to which the contrast agent is administered, with respect to the test administration performed by the contrast agent injector, and
the judging unit judges whether or not such the test administration included in the injection protocol or a test scan included in the image taking protocol that has same conditions regarding the type of the contrast agent and the site to which the contrast agent is administered, with respect to either has already been performed.

5. The image diagnosis apparatus according to claim 3, wherein, when the image taking process performed on the subject is finished, the storage unit erases the stored information indicating that the test scan has already been performed.

6. The image diagnosis apparatus according to claim 4, wherein, when the image taking process performed on the subject is finished, the storage unit erases the stored information indicating that the test scan has already been performed.

7. An image diagnosis apparatus comprising:
a judging unit configured to judge whether an image taking protocol related to an image taking process performed on a subject and an injection protocol related to the administration of the contrast agent to the subject are consistent with each other, the injection protocol being set by a contrast agent injector configured to administer a contrast agent to the subject; and
a protocol correcting unit configured, when it has been determined that the injection protocol and the image taking protocol are not consistent with each other, to correct one of the injection protocol and the image taking protocol so as to match the other.

8. The image diagnosis apparatus according to claim 7, wherein
the injection protocol includes a presence/absence of a test administration,
the image taking protocol includes a presence/absence of a test scan corresponding to the test administration, and
the judging unit judges whether the injection protocol and the image taking protocol are consistent with each other, on a basis of the presence/absence of the test administration in the injection protocol and the presence/absence of the test scan corresponding to the test administration in the image taking protocol.

9. The image diagnosis apparatus according to claim 8, wherein, when it has been determined that the injection protocol and the image taking protocol are not consistent with each other, the protocol correcting unit corrects one of the presence/absence of the test administration in the injection protocol and the presence/absence of the test scan corresponding to the test administration in the image taking protocol so as to match the other.

10. The image diagnosis apparatus according to claim 8, further comprising: a storage unit configured, when the test scan corresponding to the test administration has been performed on the subject, to store therein information indicating that the test scan has already been performed, wherein
when the injection protocol includes the test administration or when the image taking protocol includes the test scan, the judging unit judges whether the test scan has already been performed or not, and
when it has been determined that the test scan has already been performed, the protocol correcting unit makes a correction so as to obtain either an injection protocol including no test administration or an image taking protocol including no test scan.

11. The image diagnosis apparatus according to claim 10, wherein
the storage unit stores therein the information indicating that the test scan has already been performed, for each of conditions regarding a type of the contrast agent and a site to which the contrast agent is administered, with respect to the test administration performed by the contrast agent injector, and
the judging unit judges whether or not such the test administration included in the injection protocol or a test scan included in the image taking protocol that has same conditions regarding the type of the contrast agent and the site to which the contrast agent is administered, with respect to either has already been performed.

12. The image diagnosis apparatus according to claim 10, wherein, when the image taking process performed on the subject is finished, the storage unit erases the stored information indicating that the test scan has already been performed.

13. The image diagnosis apparatus according to claim 11 wherein, when the image taking process performed on the subject is finished, the storage unit erases the stored information indicating that the test scan has already been performed.

14. A contrast agent injector comprising:
a judging unit configured to judge whether an injection protocol related to an administration of a contrast agent to a subject and an image taking protocol related to the image taking process performed on the subject are consistent with each other, the image taking protocol being set by an image diagnosis apparatus configured to perform an image taking process on the subject; and
an output controlling unit configured, when it has been determined that the injection protocol and the image taking protocol are not consistent with each other, to cause a predetermined output unit to output information indicating that the injection protocol and the image taking protocol are not consistent with each other.

15. A contrast agent injector comprising:
a judging unit configured to judge whether an injection protocol related to an administration of a contrast agent to a subject and an image taking protocol related to the image taking process performed on the subject are consistent with each other, the image taking protocol being set by an image diagnosis apparatus configured to perform an image taking process on the subject; and
a protocol correcting unit configured, when it has been determined that the injection protocol and the image taking protocol are not consistent with each other, to correct one of the injection protocol and the image taking protocol so as to match the other.

16. A contrast enhanced imaging aiding apparatus comprising:
a judging unit configured to judge whether an image taking protocol related to the image taking process performed on the subject and an injection protocol related to the administration of the contrast agent to the subject are consistent with each other, the image taking protocol being set by an image diagnosis apparatus configured to perform an image taking process on a subject, and the injection protocol being set by a contrast agent injector configured to administer a contrast agent to the subject; and
an output controlling unit configured, when it has been determined that the injection protocol and the image taking protocol are not consistent with each other, to cause a predetermined output unit to output information indicating that the injection protocol and the image taking protocol are not consistent with each other.

17. A contrast enhanced imaging aiding apparatus comprising:
a judging unit configured to judge whether an image taking protocol related to the image taking process performed on the subject and an injection protocol related to the administration of the contrast agent to the subject are consistent with each other, the image taking protocol being set by an image diagnosis apparatus configured to perform an image taking process on a subject, and the injection protocol being set by a contrast agent injector configured to administer a contrast agent to the subject; and
a protocol correcting unit configured, when it has been determined that the injection protocol and the image taking protocol are not consistent with each other, to correct one of the injection protocol and the image taking protocol so as to match the other.

18. An image diagnosis system comprising: a contrast agent injector configured to administer a contrast agent to a subject; and an image diagnosis apparatus configured to perform an image taking process on the subject, wherein
the image diagnosis apparatus includes:
a judging unit configured to judge whether an image taking protocol related to the image taking process performed on the subject and an injection protocol related to the administration of the contrast agent to the subject are consistent with each other, the injection protocol being set by the contrast agent injector configured to administer the contrast agent to the subject; and
an output controlling unit configured, when it has been determined that the injection protocol and the image taking protocol are not consistent with each other, to cause a predetermined output unit to output information indicating that the injection protocol and the image taking protocol are not consistent with each other.

19. An image diagnosis system comprising: a contrast agent injector configured to administer a contrast agent to a subject; and an image diagnosis apparatus configured to perform an image taking process on the subject, wherein
the image diagnosis apparatus includes:
a judging unit configured to judge whether an image taking protocol related to the image taking process performed on the subject and an injection protocol related to the administration of the contrast agent to the subject are consistent with each other, the injection protocol being set by the contrast agent injector configured to administer the contrast agent to the subject; and
a protocol correcting unit configured, when it has been determined that the injection protocol and the image taking protocol are not consistent with each other, to correct one of the injection protocol and the image taking protocol so as to match the other.
